(19) United States
(12) Patent Application Publication
(10) Pub. No.: US 2006/0093604 A1
(43) Pub. Date: May 4, 2006

(54) Method of treatment and prophylaxis

(75) Inventors: Kathryn Elizabeth Lawlor, Victoria (AU); Ian Peter Wicks, Victoria (AU); Ian Keith Campell, Victoria (AU); Andrew Warwick Roberts, Victoria (AU); Donald Metcalf, Victoria (AU)

Correspondence Address: SCULLY, SCOTT, MURPHY & PRESSER, 400 GARDEN CITY PLAZA, SUITE 300, GARDEN CITY, NY, 11530, US

(73) Assignee: The Walter and Eliza Hall Institute of Medical Research, Parkville, Victoria (AU)

(21) Appl. No.: 10/525,363

(22) PCT Filed: Aug. 22, 2003
(86) PCT No.: PCT/AU03/01078

§ 371 Date: Sep. 6, 2005

(30) Foreign Application Priority Data
Aug. 23, 2002 (AU) 2002950957
Aug. 29, 2002 (AU) 2002951071

(51) Int. Cl. A61K 48/00; A61K 39/395; A61K 38/17

(52) U.S. Cl. 424/145.1; 514/12; 514/44

(57) ABSTRACT

The present invention relates generally to a method for treating or preventing or otherwise ameliorating the effects of inflammatory conditions such as but not limited to chronic immune-mediated inflammatory diseases. The present invention further provides pharmaceutical compositions comprising agents which inhibit one or more inflammatory cytokines and/or which down-regulate expression of genes which encode inflammatory cytokines. Such compositions are useful in the treatment and prophylaxis of inflammatory conditions such as inflammatory arthritis amongst other chronic immune-mediated inflammatory diseases. The present invention further provides an animal model for studying the kinetics of and/or screening for agents useful in the treatment or prophylaxis of inflammatory conditions.

### FIELD OF THE INVENTION

The present invention relates generally to a method for treating or preventing or otherwise ameliorating the effects of inflammatory conditions such as but not limited to chronic immune-mediated inflammatory diseases. The present invention further provides pharmaceutical compositions comprising agents which inhibit one or more inflammatory cytokines and/or which down-regulate expression of genes which encode inflammatory cytokines. Such compositions are useful in the treatment and prophylaxis of inflammatory conditions such as inflammatory arthritis amongst other chronic immune-mediated inflammatory diseases. The present invention further provides an animal model for studying the kinetics of and/or screening for agents useful in the treatment or prophylaxis of inflammatory conditions.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Granulocyte colony-stimulating factor (G-CSF, encoded by the CSF-3 gene) is a hematopoietic growth factor that regulates the production of granulocytes (Nicola et al., Nature 314: 625, 1985; Metcalf, International Journal of Cancer 25: 225, 1980; Nicola et al., Journal of Biological Chemistry 258: 9017, 1983). G-CSF mediates its effects through interaction with the granulocyte-colony stimulating factor receptor (G-CSFR, encoded by the CSFR-3 gene), a member of the type I cytokine receptor superfamily (Demetri et al., Blood 78: 2791-2808, 1991). Major biological actions of G-CSF in humans and mice, include increasing the production and release of neutrophils from the bone marrow (Souza et al., Science 232: 61, 1986; Lord et al., Proc. Natl. Acad. Sci. USA 86:9499-9503, 1989), mobilizing hematopoietic progenitor cells from the marrow into the peripheral blood (Bungart et al., British Journal of Haematology 22: 1156, 1990; de Haan et al., Blood 86: 2986-2992, 1995; Roberts et al., Blood 89: 2736-2744, 1997), and modulating the differentiation and effector functions of mature neutrophils (Yong et al., European Journal of Haematology 49: 251-259, 1992; Colotta et al., Blood 80: 2012-2020, 1992; Rex et al., Transfusion 35: 605-611, 1995; Gericke et al., Journal of Leukocyte Biology 57: 455-461, 1995; Xu et al., British Journal of Haematology 93: 558-568, 1996; Yong, British Journal of Haematology 94: 4047, 1996; Jacob et al., Blood 92: 353-361, 1998). G-CSF is used to treat neutropenia, as well as mobilisation of haemopoietic stem cells (HSC) for autologous and allogenic stem cell transplantation (Welte et al., Blood 88: 1907-1929, 1996).

Use of G-CSF for HSC mobilization can cause exacerbations of rheumatoid arthritis (RA) (Snowden et al., Bone Marrow Transplantation 22: 1035-1041, 1998). G-CSF along with colony stimulating factors, GM-CSF and M-CSF are produced by human synovial fibroblasts and chondrocytes in response to IL-1 and TNF in vitro (Leizer et al., Blood 76: 1989-1996, 1990; Hamilton et al., Blood 79: 1413-1419, 1992), and G-CSF has been found in the serum and synovial fluid of RA patients (Tanabe et al., Rheumatology International 16: 67-76, 1996; Nakamura et al., Clinical and Experimental Rheumatology 18: 713-718, 2000). Systemic administration of G-CSF has been shown to exacerbate murine collagen-induced arthritis (CIA) with increased severity and incidence of disease in DBA/1 mice (Campbell et al., Journal of Leukocyte Biology 68: 144-150, 2000), as well as a passive transfer model of CIA in rats (Miyahara et al., Clinical Immunology and Immunopathology 69: 69-76, 1993). G-CSF transgenic mice have increased bone resorption and reduced bone formation (Takahashi et al., Laboratory Investigation 74:827-834, 1996), indicating that G-CSF may have a role in bone turnover.

G-CSF is able to expand a monocyte/macrophage subset and induce anti-inflammatory cytokines that can protect against endotoxemia in mice (Gorgen et al., Journal of Immunology 149: 918, 1992). G-CSF has also been reported to impair allogeneic and mitogenic T cell responses in both humans and mice (Foster et al., Transplantation 59: 1557, 1995; Pan et al., Blood 86: 4422, 1995), and to cause a shift of the T cell cytokine profile towards Th2 cytokine production, with a corresponding reduction in Th1 IFN-γ expression (Pan et al., 1995, supra; Franzke et al., Blood 102: 734-739). In murine studies, this deviation to Th2 cytokine production has been associated with protection against acute graft-versus-host disease, experimental autoimmune encephalomyelitis (EAE) and spontaneous systemic lupus erythematosus (Pan et al., 1995, supra; Zavala et al., Journal of Immunology 163: 5125-5132, 1999; Zavala et al., Journal of Immunology 168: 2011-2019, 2000). Mice deficient in G-CSF were protected from neutrophil-mediated glomerulonephritis, but not T cell/macrophage-mediated glomerulonephritis (Kitching et al., Journal of the American Society of Nephrologh 13: 350-358, 2000).

G-CSF is, therefore, a pleiotropic molecule with a range of functions. There is a need to more fully characterize these functions and to elucidate if modulation of these functions can lead to health benefits.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The role of neutrophils was studied in a murine model of arthritis. The murine model included use of antibodies to deplete neutrophils as well as the use of a G-CSF knockout mouse. It was determined that these mice were highly resistant to the induction of acute arthritis, but this effect did not seem to be explained by the lower neutrophil counts. It was similarly found that G-CSF could directly induce joint inflammation by local administration and that systemic G-CSF could substitute for IL-1 in the model of acute arthritis.

Collagen induced arthritis (CIA) is a chronic autoimmune model widely used to investigate rheumatoid arthritis (RA) pathogenesis and for evaluation of prospective therapies. To examine the requirement for endogenous G-CSF in chronic joint disease, G-CSF^{-/-} and wild-type (WT) mice were immunized with chick Type II collagen (CII) in Complete Freund's Adjuvant (CFA) to induce CIA. There was marked protection from disease in mice deficient in G-CSF, identifying a major role for G-CSF in CIA. T cell responses to CII were normal in G-CSF knockout mice.

Collectively, this shows that endogenous G-CSF plays a major role in inflammatory arthritis. Down-regulating G-CSF activity, locally or systemically or reducing levels of G-CSF or inhibiting or down-regulating the G-CSF receptor (G-CSFR), is proposed to be a useful mechanism to treat or reduce the severity of inflammatory conditions such as chronic inflammatory arthritis and rheumatoid arthritis or other chronic immune-mediated inflammatory disease conditions.

Accordingly, the present invention contemplates a method for the prophylaxis and/or treatment of an inflammatory condition by administering to a subject an agent which inhibits the activity of, or reduces the level of, an inflammatory cytokine such as but not limited to G-CSF or its functional equivalents or homologs or its receptor (G-CSFR).

The therapeutic efficacy of administration of a neutralising monoclonal antibody (mAb) to G-CSF was tested in the acute arthritis model. Inhibition of G-CSF during acute arthritis resulted in a dose dependent reduction in myeloid lineage cells in the BM, neutropenia in the peripheral blood and reduced cellular infiltration of the involved joint.

The present invention further provides agents and pharmaceutical compositions comprising such agents which inhibit the activity of or down-regulate expression of a gene encoding G-CSF and/or other inflammatory cytokines and/or their receptors.

The present invention further provides an animal model for studying chronic inflammation and its use in screening for agents useful in the treatment and/or prophylaxis of an inflammatory condition such as inflammatory arthritis.

A list of abbreviations used herein is provided in Table 1.

TABLE 1
Abbreviations

| ABBREVIATION | DESCRIPTION |
|---|---|
| acute arthritis | methylated bovine serum albumin/IL-1-induced |
| | arthritis |
| Ab | antibody |
| B6 | C57BL/6 |
| BM | bone marrow |
| BSA | bovine serum albumin |
| CFA | Complete Freund's Adjuvant |
| CIA | Collagen-induced arthritis |
| CII | Type II collagen |
| CSF3/Csf3 | Colony stimulating factor 3 gene (human/mouse) |
| CSF3R | Colony stimulating factor 3 receptor gene (human) |
| DNP | [dinitrophenyl] acetyl |
| EAE | Experimental autoimmune encephalomyelitis |
| FACS | fluorescence activated cell sorting |
| FCS | foetal calf serum |
| FITC | fluorescein isothiocyanate |
| G-CSF | granulocyte colony-stimulating factor |
| G-CSF^{-/-} | granulocyte colony-stimulating factor-deficient |
| G-CSFR | granulocyte colony-stimulating factor receptor |
| GM-CSF | granulocyte-macrophage colony stimulating factor |
| H&E | hemotoxylin and eosin |
| HRP | horse radish peroxidase |
| HSC | haemopoietic stem cell |
| i.a. | intra-articular (ly) |
| i.d. | intra-dermal (ly) |
| Ig | immunoglobulin |
| IL- | interleukin- |
| IFN-γ | interferon-gamma |
| i.p. | intra-peritoneal (ly) |
| i.v. | intra-venous |
| KLH | keyhole limpet cyanin |
| LN | lymph node |
| mAb | monoclonal antibody |
| mBSA | methylated bovine serum albumin |
| mBSA/ | methylated bovine serum albumin-induced arthritis |
| IL-1-induced | |
| arthritis | |
| M-CSF | macrophage colony stimulating factor |
| M-CSFR | macrophage colony stimulating factor receptor |
| NMS | normal mouse serum |
| NP- | [4-hydroxy-3-nitrophenyl] acetyl |
| PBS | phosphate buffered saline |
| PE | phycoerythrin |
| RA | Rheumatoid arthritis |
| rHuG-CSF | recombinant human G-CSF |
| (filgrastim) | |
| s.c. | subcutaneous (ly) |
| TdR | thymidine |
| TNF | tumour necrosis factor |
| TNP | trinitrophenyl |
| WEHI | The Walter and Eliza Hall Institute of Medical |
| | Research |
| WT | wild type |

### BRIEF DESCRIPTION OF THE FIGURES

FIG. 1 is a graphical representation showing intra-articular effects of G-CSF. A. Mice were treated intra-articularly with filgrastim (G-CSF; 0.1, 0.5, or 1 fig), IL-1 (25 ng) or saline (vehicle) for three consecutive days and examined histologically at day 3. * P<0.05; † P<0.005 compared to saline control. B. Joint exudate cells in G-CSF (0.5, 1.5 μg) or IL-1 injected joints were quantified. There was a prominent monocyte/macrophage exudate in the G-CSF-injected joints. n>12 joints per group.

FIGS. 2A and B are graphical representations showing that G-CSF is necessary for local IL-1 induced joint inflammation, but not IL-1 induced proteoglycan loss. B6 and G-CSF−/− mice were injected i.a. with IL-1 (25 ng) on days 0, 1 and 2, and assessed histologically at day 3 for A. the severity of inflammatory and destructive features and B. loss of articular cartilage proteoglycan. Results are representative of the mean±SEM (scored out of 5). Data are from 1 of 2 experiments; n=14 joints/group; * p<0.05.

FIGS. 3A and 3B show graphical representations of the systemic effects of filgrastim in lieu of IL-1 in the acute mBSA/IL-1-induced model. B6 mice were injected i.a. with mBSA and s.c. on days 0, 1, 2 with saline [black bars], IL-1 (250 ng) [grey bars] or filgrastim (15 μg) [white bars]. Shown are A. Peripheral blood counts at day 2 and B. popliteal lymph node (LN) and spleen weights at day 7. Data are representative of 3 experiments with n>5 mice per group. * P<0.05; † P<0.01 compared to mBSA/saline control group.

FIGS. 4A and 4B illustrate the effects of systemic G-CSF in lieu of IL-1 in the acute arthritis model. Mice were injected i.a. with mBSA and s.c. with either saline vehicle, IL-1, or G-CSF and the injected knees were examined histologically at day 7. A. Mean total histological scores ±SEM. B Representative histology illustrating i mBSA/saline treated joint with minimal exudate cells in the joint space (100×), ii mBSA/IL-1-treated joint with moderate to marked exudate and synovitis and iii mBSA/G-CSF-treated joint with moderate inflammatory features (200×). n≧10 joints/group/experiment; representative experiment of three. * p<0.05 compared to mBSA/saline controls.

FIG. 5A-C G-CSF−/− mice are relatively resistant to mBSA/IL-1-induced arthritis. B6 and G-CSF−/− mice were treated i.a. with mBSA and s.c. with IL-1 or saline and histologically assessed at day 7. Histograms illustrate A Total histological severity scores and B cartilage proteoglycan loss (by safranin O staining). C Representative sections showing H&E (top panel) and safranin O (bottom panel) sections from mBSA/IL-1-treated (i, iii) B6 and (ii, iv) G-CSF−/− mice. n>6 joints/group/experiment; representative of 3 experiments.

FIG. 6 shows representative FACS plots of leukocyte populations infiltrating the synovial tissue from B6 mBSA/saline, B6 mBSA/IL-1 and G-CSF−/− mBSA/IL-1 treated mice at days 3 and 7. Synovium was dissected at A day 3 and B day 7 from greater than 6 joints/group, dissociated in an enzyme cocktail and then stained for specified markers. In A & B, total infiltrating leukocytes were identified by staining with CD45.2 (A,B; top panel). Only the CD45.2+ population was used for subsequent analyses. C Synovial digests from B6 mBSA/saline, B6 mBSA/IL-1 and G-CSF−/− mBSA/IL-1 treated mice were also adhered overnight and non-adherent cells stained for CD4 expression. Data are representative of 2 experiments.

FIGS. 7A and 7B are graphical representations showing the effects of neutrophil depletion in acute arthritis in WT B6 and G-CSF−/− mice. Mice were treated prior to mBSA/IL-1-induced arthritis induction with a depleting mAb to neutrophils or isotype control. A Peripheral blood analysis on days 0, 2 and 7 of the acute arthritis model in WT and G-CSF−/− mice treated with neutrophil-depleting mAb or isotype control mAb. B Total histological scores of WT and G-CSF−/− mice treated with anti-neutrophil mAb or isotype control. n>5 joints per group * P<0.05 compared to WT isotype control mAb-treated mice neutrophil levels. † P<0.05 compared to WT isotype control and anti-neutrophil mAb-treated control group total scores.

FIG. 8 graphical representations showing impaired CIA in G-CSF−/− mice compared to B6 mice. Mice were injected intra-dermally at the base of the tail with CII in CFA and boosted at day 21. Mice were clinically assessed for disease from day 21 with each paw being scored from 0 (normal) to three (severe); maximal score 12 (For details see EXAMPLE 8). A. illustrates the cumulative incidence of disease. B. The clinical severity of CIA in B6 and G-CSF−/− mice. Data are pooled from 3 experiments; n>30 mice per group. * P<0.001 B6 compared to G-CSF−/−.

FIG. 9 shows results of histological assessment of CIA in B6 versus G-CSF−/− mice. Joints from four of the most severely clinically affected B6 and G-CSF−/− mice were scored from 0 to 3 for histopathology severity. A is a graphical representation of the percentage of normal, mild, moderate and severely affected joints. B shows representative H&E sections from i a non-arthritic B6 joint, ii a severely inflamed B6 CIA joint and iii a typical G-CSF−/− joint that exhibits no inflammation.

FIG. 10 are graphical representations showing T cell responses to CII in vitro in B6 and G-CSF−/− mice. Single inguinal LN suspensions were plated and stimulated with denatured CII. Cells were pulsed for the last 8 h with [³H] TdR and radioactive uptake measured to assess T cell proliferation. A. Proliferative stimulation index in B6 and G-CSF−/− LN cells. B. Supernatants taken from cultures at 64 h were assayed for levels of (i) IFN-γ and (ii) IL-2 by ELISA. n>6 wells/sample.

FIGS. 11A and B show anti-CII Abs in CIA-immunised B6 and G-CSF−/− mice. Serum was taken at A. day 30 and B. day 62 and analysed by ELISA for anti-CII Abs-total IgG, IgM and isotypes IgG2b, IgG2c, IgG1 and IgG3. Data are pooled from 3 experiments; n>30 samples per group. * P<0.05, † P<0.005.

FIG. 12 shows graphical representation of basal Ig levels and levels of non-specific total IgG in naive and CII/CFA-immunised B6 and G-CSF−/− mice. A. Naïve B6 and G-CSF−/− mice (n=6 mice/group) were bled and sera tested by ELISA for levels of circulating total IgG, IgM and isotypes (IgG2b, IgG2c, IgG1, IgG3 and IgA). B. Day 62 sera from CIA-immunised B6 and G-CSF−/− mice was analysed for non-specific total IgG. Mice from 3 separate experiments are included; n≧16 mice/group; * P<0.05.

FIG. 13 shows Ab responses to T-dependent and T-independent antigens in G-CSF−/− mice compared to B6 mice. B6 and G-CSF−/− mice were challenged with the T-dependent Ag NP-KLH precipitated in alum or with the T-independent antigen DNP-Dextran in PBS and bled on specified days. The NP-KLH group were boosted at day 42. Sera were analysed by ELISA for assessment of A. T-dependent NP-KLH response as assessed by the titres of i NP₂ (total) and ii NP₂₀ (high affinity)-specific IgG1, iii NP₂₀ Ig2b and B T-independent DNP-dextran anti-NP IgM. n>4 mice per group.

FIG. 14 shows effects of G-CSF neutralisation in the acute arthritis model. B6 mice were treated with neutralising anti-G-CSF (50 and 250 μg) or isotype control mAb on days 0, 1, 2, 3 and 5. Graphical representations of A. peripheral blood neutrophil counts at days 4 and 7 (n=5) B. Mean clinical macroscopic score per joint (out of 4) (n=10) C. mean synovial tissue cellularity per mouse joint (n=3). D. Dot plots showing infiltrating neutrophils (GR1hi CD11bhi) and monocyte/macrophages (CD11bhi GR1lo). Data are representative of a single experiment; * P<0.01.

### DETAILED DESCRIPTION OF THE PREFERRED EMBODIMENTS

The present invention is predicated in part on the further elucidation of the role of inflammatory cytokines in the inflammatory process. More particularly, the role of G-CSF is postulated to have an effect on inflammatory conditions such as chronic immune-mediated inflammation. In accordance with the present invention, therefore, inhibiting the activity of the inflammatory cytokine locally or systemically and/or down-regulating expression of a gene encoding an inflammatory cytokine is proposed to be useful in the treatment or prophylaxis of an inflammatory condition.

Accordingly, one aspect of the present invention contemplates a method for the treatment or prophylaxis of an inflammatory condition in an animal or avian species, said method comprising administering to said animal or avian species an effective amount of an agent which inhibits the activity of an inflammatory cytokine or its receptor and/or which reduces the level of expression of the gene encoding said inflammatory cytokine or its receptor.

The present invention is particularly directed to G-CSF and its homologs and derivatives. Reference to “G-CSF” or its full name “granulocyte-colony stimulating factor” includes it homologs and derivatives. A “homolog” or “derivative” includes polymorphic variants of G-CSF. Reference herein to G-CSF may also be read as applying to other inflammatory cytokines.

Accordingly, another aspect of the present invention provides a method for the treatment and/or prophylaxis of an inflammatory condition in an animal or avian species, said method comprising administering to said animal an agent which inhibits the activity of G-CSF and/or which reduces the level of expression of the gene encoding G-CSF or G-CSFR.

As indicated above, reference to “G-CSF” includes its homologs and it derivatives.

The administration may be systemic or local. Local administration is particularly useful in the treatment of localized or inflammatory conditions such as arthritis. However, as it is likely that G-CSF exerts effects on haemopoietic cells, systemic administration may be useful in modulating the immune system in general. Reference to “systemic” includes intra-articular, intravenous, intraperitoneal, subcutaneous and intrathecal administration as well as administration via oral, rectal and nasal routes.

The term “inflammatory condition’ is used in its broadest context but particularly encompasses immune system-mediated inflammatory condition. In a particularly important embodiment, the inflammatory condition is inflammatory arthritis including rheumatoid arthritis (RA).

Accordingly, in a preferred embodiment, the present invention contemplates a method for the treatment and/or prophylaxis of an inflammatory arthritis or other chronic immune-mediated inflammatory condition in an animal or avian species, said method comprising administering to said animal or avian species an agent which inhibits the activity of G-CSF or G-CSFR and/or which reduces the level of expression of the gene encoding G-CSF or G-CSFR.

The preferred animals are mammals such as humans and other primates, livestock animals (e.g. sheep, cows, horses, donkeys, pigs), laboratory test animals (e.g. rabbits, mice, hamsters, guinea pigs), companion animals (e.g. dogs, cats) and captive wild animals. Avian species include poultry birds (e.g. chickens, ducks, geese, turkeys, bantams), game birds (e.g. ducks, emus, pheasants) and caged avian birds. Humans are the most preferred animals of the primates. Horses are particularly preferred of the livestock animals.

In a preferred embodiment, therefore, the present invention provides a method for the treatment and/or prophylaxis of an inflammatory condition in a human, said method comprising administering to said human an agent which inhibits the activity of G-CSF or G-CSFR and/or which reduces the level of expression of the gene encoding G-CSF or G-CSFR.

The agents may be proteinaceous, non-proteinaceous (e.g. chemical entities) or nucleic acid molecules.

Proteinaceous and non-proteinaceous molecules include peptides, polypeptides and proteins, small, intermediate or large chemical molecules as well as molecules identified from natural product screening or the screening of chemical libraries. Natural product screening includes the screening of extracts or samples from plants, microorganisms, soil river beds, coral, aquatic environments and extraterrestrial environments for molecules or groups of molecules which have an affect on G-CSF activity or the level of G-CSF gene expression. These molecules may also affect G-CSF/G-CSFR interaction.

One example of an agent is an antibody to G-CSF or G-CSFR or epitopes thereon. This could be used systemically or locally.

The use of monoclonal antibodies is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976).

Another example of a useful agent is a soluble form of the G-CSFR which competes with G-CSF interaction with the membrane-associated G-CSFR.

Alternatively, agents can be screened for their ability to bind to G-CSF or G-CSFR-genetic materials. In one embodiment, G-CSF- or G-CSFR-encoding cDNA or genomic DNA or mRNA transcript or portion thereof such as an EST or SAGE tag is immobilized to a solid support such as a nanoparticle or microsphere. Potential agents are then brought into contact with the immobilized nucleic acid molecules and binding detected by change in radiation, emissions, atom excitation, mass and/or density.

Once identified, the agent is eluted off the nucleic acid molecule and characterized in more detail. For example, agents which bind to G-CSF/G-CSFR genetic material may inhibit expression (transcription and/or translation).

The present invention further contemplates using chemical analogs of G-CSF or G-CSFR as antagonists of G-CSF or its receptors. As indicated above, soluble G-CSF receptors may also be employed.

Chemical analogs contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homobifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Nucleic acid molecules such as RNA or DNA are particularly useful for inducing gene silencing by antisense- or sense-mediated mechanisms. Sense-mediated gene silencing is also referred to as co-suppression and involves a range of mechanisms including the induction of RNAi.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts of the G-CSF genetic sequence or the G-CSFR genetic sequence. Furthermore, polynucleotide vectors containing all or a portion of the G-CSF gene locus may be placed under the control of a promoter in either the sense or antisense orientation and introduced into a cell. Expression of such a sense or antisense construct within a cell interferes with target transcription and/or translation. Furthermore, co-suppression (i.e. using sense-suppresion) and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules encoding G-CSF or G-CSFR, i.e. the oligonucleotides induce transcriptional or post-transcriptional gene silencing. This is accomplished by providing oligonucleotides which specifically hybridize with one or more nucleic acid molecules encoding the inhibitor. The oligonucleotides may be provided directly to a cell or generated within the cell. As used herein, the terms “target nucleic acid” and “nucleic acid molecule encoding G-CSF or G-CSFR” have been used for convenience to encompass the encoding DNA, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those herein described.

The open reading frame (ORF) or “coding region” which is known in the art to refer to the region between the translation initiation codon and the translation termination codon, is a region which may be effectively targeted. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

For topical delivery of antisense compounds, these oligonucleotides may contain modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In an alternative embodiment, genetic constructs including DNA “vaccines” are used to generate antisense or sense molecules mammalian cells. Furthermore, many of the preferred features described above are appropriate for sense nucleic acid molecules.

Agents identified in accordance with the present invention are conveniently supplied in pharmaceutical compositions.

The present invention further contemplates a composition comprising a modulator of G-CSF activity or the interaction between G-CSF and G-CSFR or a modulator of expression of G-CSF/G-CSFR, said composition further comprising one or more pharmaceutically acceptable carriers and/or diluents.

Composition forms suitable for injectable use include sterile aqueous solutions (where water soluble) and sterile powders for the extemporaneous preparation of sterile injectable solutions. It must be stable under the conditions of manufacture and storage and must be preserved against the contaminating action of microorganisms such as bacteria and fungi. The carrier can be a solvent or dilution medium comprising, for example, water, ethanol, polyol (for example, glycerol, propylene glycol and liquid polyethylene glycol, and the like), suitable mixtures thereof and vegetable oils. The proper fluidity can be maintained, for example, by the use of superfactants. The preventions of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization. In the case of sterile powders for the preparation of sterile injectable solutions, suitable methods of preparation include vacuum drying and the freeze-drying technique which yield a powder of active ingredient plus any additionally desired ingredient.

When the modulator is suitably protected, it may be orally administered, for example, with an inert diluent or with an assimilable edible carrier, or it may be enclosed in hard or soft shell gelatin capsule, or it may be compressed into tablets, or it may be incorporated directly with the food of the diet or administered via breast milk. For oral therapeutic administration, the active ingredient may be incorporated with excipients and used in the form of ingestible tablets, buccal tablets, troches, capsules, elixirs, suspensions, syrups, wafers and the like. Such compositions and preparations should contain at least 1% by weight of modulator. The percentage of the compositions and preparations may, of course, be varied and may conveniently be between about 5 to about 80% of the weight of the unit. The amount of modulator in such therapeutically useful compositions is such that a suitable dosage will be obtained. Preferred compositions or preparations according to the present invention are prepared so that an oral dosgae unit form contains between about 0.1 μg and 200 mg of modulator. Alternative dosage amounts include from about 1 μg to about 1000 mg and from about 10 μg to about 500 mg. These dosages may be per individual or per kg body weight. Administration may be per hour, day, week, month or year.

The tablets, troches, pills, capsules, creams and the like may also contain the components as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the modulator, their use in the therapeutic compositions is contemplated. Supplementary active compounds can also be incorporated into the compositions.

As indicated above, administration may be by any means. For the treatment of arthritis or local inflammations, intra-articular or subcutaneous administration is particularly preferred.

The composition may also comprise genetic molecules such as a vector capable of transfecting target cells where the vector carries a nucleic acid molecule capable of encoding a modulator, when the modulator is a proteinaceous molecule. The vector may, for example, be a viral vector. In this regard, a range of gene therapies are contemplated by the present invention including isolating certain cells, genetically manipulating and returning the cell to the same subject or to a genetically related or similar subject.

The present invention further provides an animal model for inflammation useful for screening for agents capable of inhibiting G-CSF or G-CSFR and thereby ameloriate the effects of inflammation. Animal models are contemplated which produce high or low levels of G-CSF or G-CSFR. Such animals are useful for screening for agents which ameliorate the symptoms of inflammation or which prevents its occurrence. Furthermore, in animals with reduced levels of G-CSF, other cytokines or endogenous molecules may emerge to compensate G-CSF's absence. These then become targes for further therapeutic molecules.

Accordingly, another aspect of the present invention provides a genetically modified animal wherein said animal produces low amounts of G-CSF or G-CSFR relative to a non-genetically modified animal of the same species.

Preferably, the genetically modified animal is a mouse, rat, guinea pig, rabbit, pig, sheep or goat. More preferably, the genetically modified animal is a mouse or rat. Most preferably, the genetically modified animal is a mouse.

Accordingly, a preferred aspect of the present invention provides a genetically modified mouse wherein said mouse produces low amounts of G-CSF or G-CSFR relative to a non-genetically modified mouse of the same strain.

The animal models of the present invention may be in the form of the animals or may be, for example, in the form of embryos for transplantation. The embryos are preferably maintained in a frozen state and may optionally be sold with instructions for use.

Yet another aspect of the present invention provides a targeting vector useful for inactivating a gene encoding G-CSF or G-CSFR, said targeting vector comprising two segments of genetic material encoding said G-CSF or G-CSFR flanking a positive selectable marker wherein when said targeting vector is transfected into embryonic stem (ES) cells and the marker selected, an ES cell is generated in which the gene encoding said G-CSF or G-CSFR is inactivated by homologous recombination.

Preferably, the ES cells are from mice, rats, guinea pigs, pigs, sheep or goats. Most preferably, the ES cells are from mice.

Still yet another aspect of the present invention is directed to the use of a targeting vector as defined above in the manufacture of a genetically modified animal substantially incapable of producing G-CSF or G-CSFR.

Even still another aspect of the present invention is directed to the use of a targeting vector as defined above in the manufacture of a genetically modified mouse substantially incapable of producing G-CSF or G-CSFR.

Preferably, the vector is DNA. A selectable marker in the targeting vector allows for selection of targeted cells that have stably incorporated the targeting DNA. This is especially useful when employing relatively low efficiency transformation techniques such as electroporation, calcium phosphate precipitation and liposome fusion where typically fewer than 1 in 1000 cells will have stably incorporated the exogenous DNA. Using high efficiency methods, such as microinjection into nuclei, typically from 5-25% of the cells will have incorporated the targeting DNA; and it is, therefore, feasible to screen the targeted cells directly without the necessity of first selecting for stable integration of a selectable marker. Either isogenic or non-isogenic DNA may be employed.

Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistance genes such as the neomycin resistance gene (neo) and the hygromycin resistance gene (hyg). Selectable markers also include genes conferring the ability to grow on certain media substrates such as the tk gene (thymidine kinase) or the hprt gene (hypoxanthine phosphoribosyltransferase) which confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). Other selectable markers for use in mammalian cells and plasmids carrying a variety of selectable markers are described in Sambrook et al., Molecular Cloning—A Laboratory Manual, Cold Spring Harbour, New York, USA, 1990.

The preferred location of the marker gene in the targeting construct will depend on the aim of the gene targeting. For example, if the aim is to disrupt target gene expression, then the selectable marker can be cloned into targeting DNA corresponding to coding sequence in the target DNA. Alternatively, if the aim is to express an altered product from the target gene, such as a protein with an amino acid substitution, then the coding sequence can be modified to code for the substitution, and the selectable marker can be placed outside of the coding region, for example, in a nearby intron.

The selectable marker may depend on its own promoter for expression and the marker gene may be derived from a very different organism than the organism being targeted (e.g. prokaryotic marker genes used in targeting mammalian cells). However, it is preferable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin). A number of other variations may be employed to enhance expression of the selectable markers in animal cells, such as the addition of a poly(A) sequence and the addition of synthetic translation initiation sequences. Both constitutive and inducible promoters may be used.

The DNA is preferably modified by homologous recombination. The target DNA can be in any organelle of the animal cell including the nucleus and mitochondria and can be an intact gene, an exon or intron, a regulatory sequence or any region between genes.

Homologous DNA is a DNA sequence that is at least 70% identical with a reference DNA sequence. An indication that two sequences are homologous is that they will hybridize with each other under stringent conditions (Sambrook et al., 1990, supra).

The present invention further contemplates co-suppression (i.e. sense suppression) and antisense suppression to down-regulate expression of G-CSF or G-CSFR. This would generally occur in a target test animal such as to generate a disease model.

The genetically modified animals may also produce larger amounts of G-CSF or G-CSFR. For example, over expression of normal G-CSF or G-CSFR may produce dominant negative effects and may become useful disease models.

Accordingly, another aspect of the present invention is directed to a genetically modified animal over-expressing a genetic sequence encoding G-CSF or G-CSFR.

A genetically modified animal includes a transgenic animal, or a “knock-out” or “knock-in” animal.

The present invention is further described by the following non-limiting Examples.

### EXAMPLE 1

### Mice

C57BL/6 (B6; wild type, [WT]) mice were obtained from the Walter and Eliza Hall Institute (WEHI) Animal Supplies (Victoria, Australia). G-CSF-deficient (G-CSF^{-/-}) mice were obtained from the Ludwig Institute for Cancer Research, Victoria, Australia and were produced by targeted disruption of the Cysf3 gene in 129/OLA embryonic stem (ES) cells, which were injected into B6 blastocysts (Lieschke et al., Blood 84: 1737-1746, 1994). Mice were backcrossed greater than twenty generations onto the B6 background. All mice were ≧8 weeks of age at the time of experimentation, were fed standard rodent chow and water ad libitum and were housed (≦6 mice/cage) in sawdust-lined cages. All animal procedures were approved by the Institutional Ethics Committee.

### EXAMPLE 2

### Induction of mBSA/IL-1-Induced Arthritis (Acute Arthritis)

The procedure was based on that previously described (Lawlor et al., Arthritis and Rheumatism 44: 442-450, 2001). Mice were anaesthetized and injected intra-articularly. into the knee joint with 10 μl of 20 mg/ml mBSA (Sigma, St Louis, Mo.). Control joints received the same volume of vehicle (normal saline). Mice were next injected subcutaneously (s.c.) into the rear footpad with 20 μl of 12.5 μg/ml recombinant human IL-1β (Specific Activity 5×10⁸ U/mg; Amgen, Thousand Oaks, Calif.) in normal saline/0.5% (v/v) normal mouse serum (vehicle) and the injection was repeated on the next 2 days.

Mice were sacrificed on day 7 (or at indicated time points), the knee joints excised and fixed in 10% (v/v) neutral-buffered formalin for at least 2 days, decalcified and processed to paraffin. Frontal tissue sections (4 μm) were cut at 4 depths approximately 100 μm apart and stained with haematoxylin and eosin (H&E) to assess joint pathology.

Assessment of arthritis was performed blinded to the experimental groups. Five components of arthritis were assessed, i.e. joint space exudate, synovitis, pannus formation, cartilage and bone degradation. These were graded for severity from 0 (normal) to 5 (severe). Based on the histological scores, joints were classified as demonstrating inflammatory arthritis if there was an exudate score of 1 or more and synovitis score of 2 or more. Destructive arthritis was classified as a score of 2 or greater for pannus and 1 or greater for cartilage and/or bone degradation. The overall mean histological severity score was also calculated, with a maximum possible score per joint of 25 (Lawlor et al., 2001, supra). Safranin O stained sections were prepared and assessed blindly for cartilage proteoglycan loss.

### EXAMPLE 3

### Induction of Collagen Induced Arthritis (CIA)

Chick type II collagen (CII; Sigma) dissolved in 10 mM acetic acid overnight at 4° C. at a concentration of 2 mg/ml, was emulsified in an equal volume of Freund's complete adjuvant (CFA), prepared at 5 mg/ml by adding heat-killed Mycobacterium tuberculosis (strain H37 Ra; Difco Laboratories, Detroit, Mich., USA) to Freund's incomplete adjuvant (Difco). Mice were injected intra-dermally (i.d.) at several sites into the base of the tail with 100 μl of the emulsion and this was repeated 21 days later.

Animals were monitored for erythema and swelling of limbs and a clinical score given to each mouse 3 times a week for up to 40 days. The scoring system was as previously described (Campbell et al., European Journal of Immunology 30: 1568-1575), where 0=normal, 1=slight swelling, 2=extensive swelling and 3=joint distortion and/or rigidity and the maximum score per mouse was 12. Clinical assessments were completed by two independent investigators blinded to the experimental groups. At sacrifice, paws were removed, fixed, decalcified and processed for paraffin embedding as described above. H&E stained sections (5 μm) of the front and rear paws of four mice with the highest clinical scores were evaluated as previously described (Campbell et al., 2000, supra). At day 30 and the day of sacrifice (day 62), blood was taken for determination of serum anti-CII Ab.

### EXAMPLE 4

### Administration of G-CSF

Intra-Articular G-CSF and IL-1

Mice received daily i.a. injection of 10 μl of IL-1 (25 ng) or recombinant human G-CSF (rHuG-CSF; 0.1, 0.5, 1 and 1.5 μg) or vehicle (saline; normal saline/0.5% (v/v) normal mouse serum (vehicle) on days 0, 1 and 2. Mice were sacrificed on day 3 and joints assessed histologically on H&E stained sections.

Subcutaneous G-CSF in Lieu of IL-1β in Acute Arthritis

Mice were injected i.a. with mBSA and treated s.c. in the footpad on days 0-2 with either IL-1 (250 ng) or rHuG-CSF [filgrastim] (15 μg) or vehicle control. Mice were sacrificed at day 7 as described above.

Depletion of Neutrophils in WT and G-CSF-Mice

WT (B6) and G-CSF^{-/-} mice were treated intra-peritoneally 2 days prior to disease induction and on days 0 to 2 with 0.6 mg neutrophil-depleting monoclonal antibody (mAb), RB6.8C5 or isotype control mAb GL121. Mice were then treated daily from days 3 to 6 with 0.5 mg of mAb. Peripheral blood was analyzed for neutrophil counts on days 0, 2 and 7 by differential cell count analysis.

### EXAMPLE 6

### T Cell Proliferation Assay

Inguinal lymph nodes (LN) were harvested from mice (n>5 mice/experiment) immunized for CIA, 52-62 days after primary injection. Single cell suspensions were prepared in RPMI containing 2-mercaptoethanol (50 μM) and 5% (vol/vol) fetal bovine serum (FCS). LN cells (2×10⁵ cells) in 200 μl were plated in a round-bottomed 96-well plate (Becton Dickinson Labware, Franklin Lakes, N.J., USA) and stimulated with 0-100 μg/ml denatured CII (boiled 10 minutes). Cells were incubated for 72 hours at 37° C. (5% CO₂), supernatants taken at 20 and 48 hours, and pulsed for the final 8 h with 1 μCi [³H] thymidine. Cells were harvested with an Inotech Cell Harvester (Inotech) and [³H] thymidine incorporation was measured as a measure of T cell proliferation using a microplate scintillation counter (Canberra Packard, Victoria, Australia). Aliquots of cell supernatants were taken at 20 and 48 hours.

Flow Cytometric Analysis of Infiltrating Leukocytes

Patellae and attached soft tissues from mBSA/saline-treated B6 mice and mBSA/IL-1-treated B6 and G-CSF−/− mice were dissected on days 3 and 7. Bone, fat and muscle fragments were discarded, the synovium was minced into 2-mm pieces and digested into a single cell suspension using 2.4 mg/ml dispase II (Boehringer, Mannheim, Germany), 1 mg/ml collagenase type II (Sigma), and 100 μg/ml DNAse I (Boehringer Mannheim, Indianapolis, USA) in RPMI 1640. The suspension was gently agitated at 37° C. for 45 min and then washed through a 70 μM nylon cell strainer (Falcon) with 10% [v/v] FCS in RPMI. Cell counts were performed prior to cell staining. Non-specific-staining was blocked using rat anti-mouse FcγRIIb/III (CD16/CD32; clone 2.4G2; American Type Culture Collection (ATCC), Manassas, Va., USA). Leukocytes were stained using biotinylated rat anti-mouse CD45.2 (PharMingen, San Diego, Calif., USA) and fluorochrome streptavidin tricolor (SA-TRI) (Caltag) and combinations of Ab listed below in EXAMPLE 12. Synovial digest cells were also adhered overnight at 37° C., 5% CO₂ to recover expression of markers such as CD4 that are cleaved by dispase. Non-adherent cells were collected in 2% v/v FCS in PBS and stained for fluorescence activated cell sorting (FACS).

### EXAMPLE 7

### Cytokine Assays and Leukocyte Morphological Quantification

IFN-γ, IL-4 and IL-2 were measured in T cell supernatants by capture ELISAs using paired monoclonal antibodies, according to the manufacturer's instructions (Pharmingen).

Joint Exudate Leukocyte Morphological Quantification

H&E stained joint sections (n=2 section depths per joint) were analysed for exudate composition by quantifying polymorphonuclear leukocytes, monocyte/macrophages and lymphocytes in 5 grid areas of focal joint space exudate at high magnification (1000×).

### EXAMPLE 8

### Determination of Serum Anti-CII Antibodies (Ab)

ELISAs were performed to detect Abs to CII as previously described (Campbell et al., 2000, supra). Horseradish peroxidase-conjugated goat anti-mouse IgG (Sigma Chemical Co.), IgG2b, IgG2c, IgG1, IgG3 or IgM (Southern Biotechnology Associates, Birmingham, Ala., USA) antisera were used as detection Abs. Standard curves were constructed from pooled sera of hyper-immunized DBA/1 mice using arbitrary units.

Determination of Serum Ig Levels

Serum Abs from retro-orbital sinus bleeds of naïve and CII/CFA-immunised mice were captured with relevant Abs to total IgG, IgM, IgG2c, IgG2b, IgG1, IgG3 and IgA and detected using horseradish peroxidase (HRP) conjugated Abs (Southern Biotechnology Associates, Birmingham, Ala., USA).

### EXAMPLE 9

### Peripheral Blood Leukocyte Counts

Peripheral blood was obtained from mice by retro-orbital plexus venesection on days 0, 3 and 7 of the acute arthritis model, and collected in EDTA coated tubes. BM was flushed from one femur per mouse into 3% [v/v] foetal calf serum (FCS)/phosphate buffered saline (PBS). Total leukocyte counts and differential analyses were performed using an Advia 120 Hematology System (Bayer Diagnostics, Tarrytown, N.Y., USA). Manual BM counts were also done on cytospins (1×10⁵ cells centrifuged onto a microscope slide at 1200 rpm for 7 min).

### EXAMPLE 10

### Joint Inflammation Develops in Response to Direct Intra-Articular Administration of G-CSF

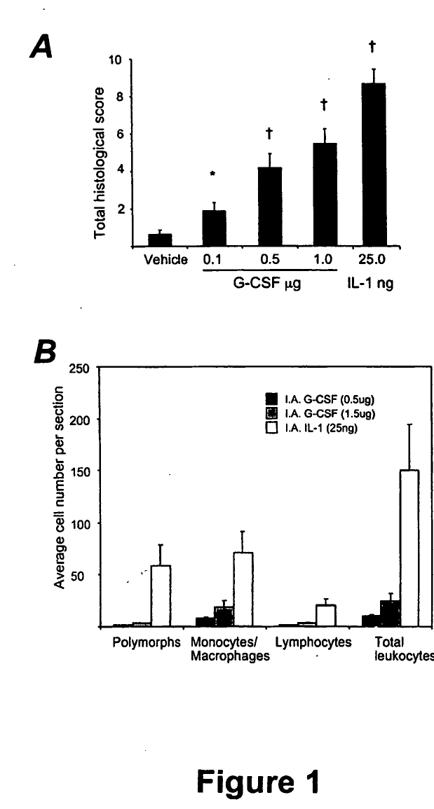

It was first investigated whether granulocyte-colony stimulating factor (G-CSF) has pro-inflammatory properties in joints by intra-articular injection of rHuG-CSF (0.1, 0.5, & 1 μg) into the knee joint of wild type (WT) C57BL/6 mice over three consecutive days. Controls included intra-articular injection of IL-1 (IL-1; 25 ng) and vehicle (0.5% [v/v] normal mouse serum in normal saline). On day 3 joints were taken for histological assessment. It was found that G-CSF induced inflammation in a dose-dependent manner (FIG. 1), although the response was significantly less than that induced with IL-1. This result shows that exogenous G-CSF has pro-inflammatory effects within the normal joint.

### EXAMPLE 11

### G-CSF-Deficient Mice Develop Less IL-1 Induced Joint Inflammation

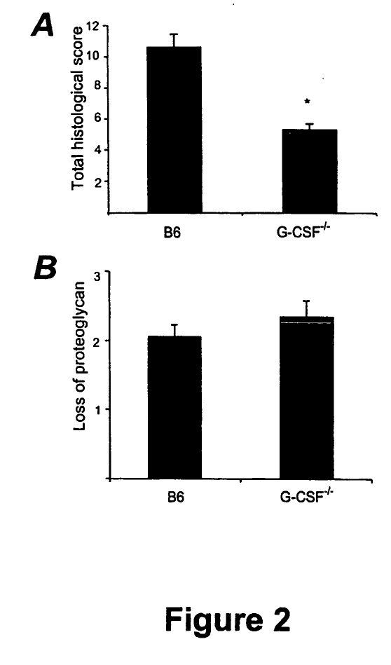

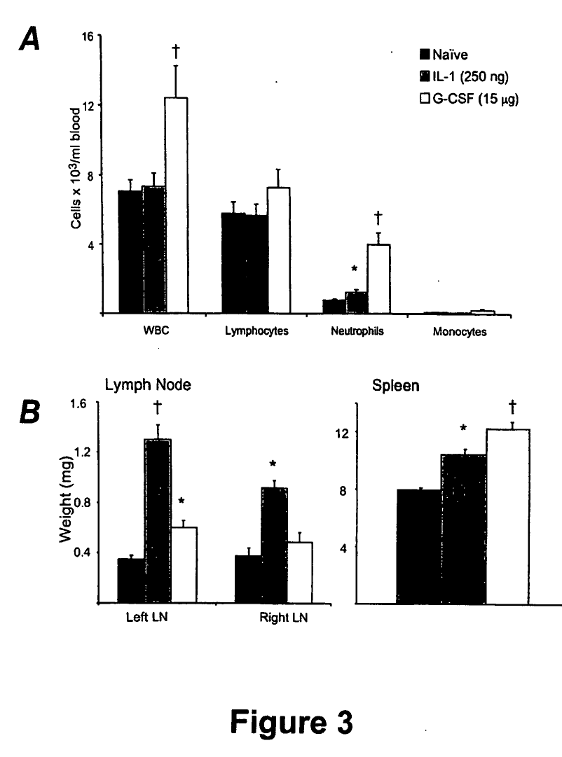

To investigate the involvement of G-CSF in modulating local IL-1-induced inflammatory effects, mice deficient in G-CSF (G-CSF−/−) and B6 mice were injected i.a. with IL-1. IL-1 induced joint inflammation in G-CSF−/− mice, but at a reduced level compared to normal B6 mice (FIG. 2A), demonstrating that G-CSF is a downstream mediator of IL-1 in the joint compartment. Despite this reduction in inflammatory features in G-CSF−/− mice, there was no difference in articular cartilage proteoglycan loss (FIG. 2B), suggesting that IL-1 can directly damage cartilage.

Peripheral Blood, BM and Synovial Infiltrate Analysis

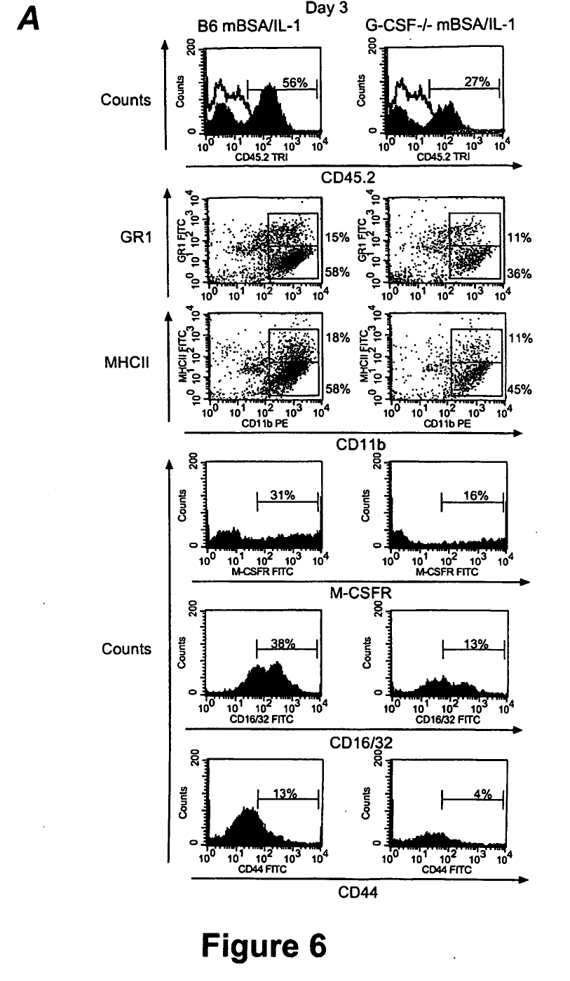

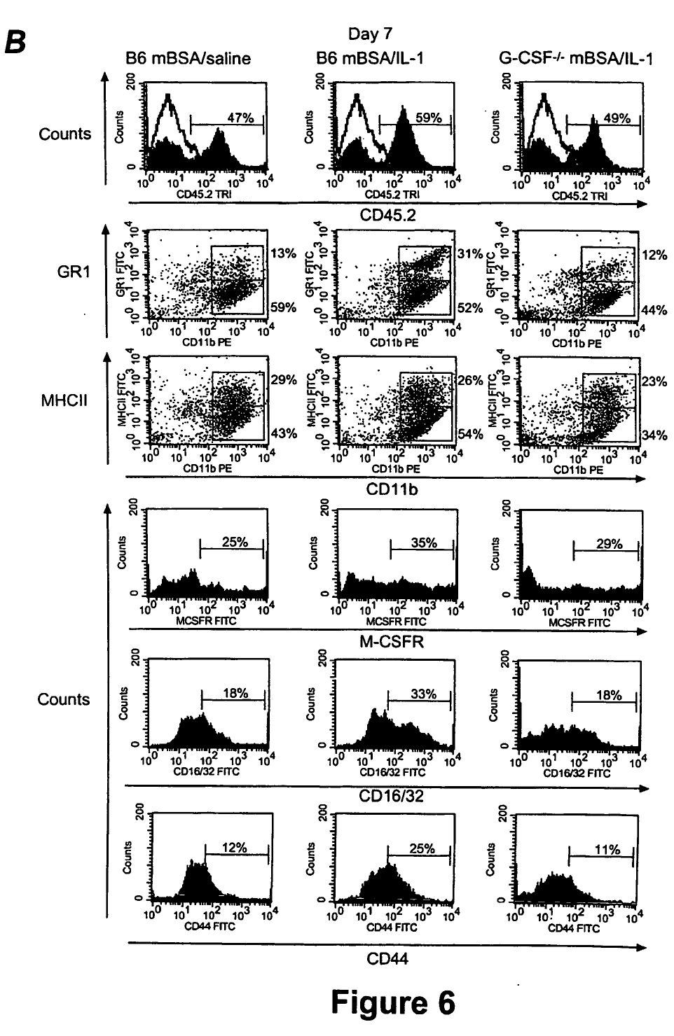

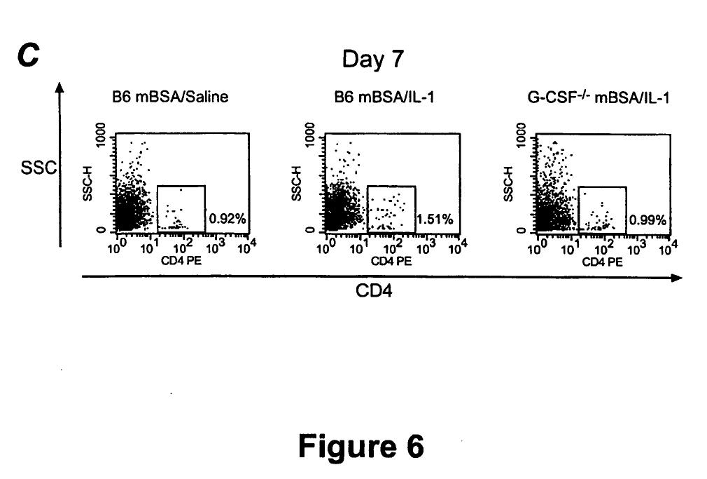

To examine the effects of G-CSF deficiency on the inflammatory response induced by mBSA and IL-1, peripheral blood, BM and synovium were taken from B6 and G-CSF−/− mice treated i.a. with mBSA and s.c. with either IL-1 or saline vehicle, at days 0, 3 and 7 of this model. Results are shown in Table 2 and 3, and in FIG. 6. G-CSF deficiency resulted in a blunted neutrophilic response to mBSA/IL-1. BM cellularity in both B6 and G-CSF−/− mice was reduced by IL-1 administration (Table 2), reflecting the mobilisation of BM cells into the blood. When compared with B6 mice, G-CSF−/− mice had significantly reduced numbers of metamyelocytes and polymorphs, as well as fewer promyelocytes and myelocytes in the BM compartment (Table 2) both basally and in response to IL-1. B6 mice developed a marked peripheral neutrophilia during the course of acute arthritis. In sharp contrast, G-CSF−/− mice exhibited a significant neutropenia over the course of the model (Table 3), indicating that the neutrophilia induced by IL-1 is G-CSF dependent

Investigation of the cellular composition of the inflamed synovium of mBSA/IL-1-treated G-CSF−/− mice also revealed a reduction in infiltrating leukocytes at day 3 and 7 (FIG. 6A-C). Cell counts of synovial tissue digests revealed approximately 2-fold reduction in total cellularity at day 7 in mBSA/IL-1-treated G-CSF−/− joint synovial tissue, compared to mBSA/IL-1-treated synovial tissue from B6 mice (1.87±0.07×10⁵ cells/B6 mBSA/saline joint versus 3.13±0.10×10⁵ cells/B6 mBSA/IL-1 joint versus 1.66±0.05×10⁵ cells/G-CSF−/− mBSA/IL-1 joint). In particular, there were significant reductions in the percentages and numbers of neutrophils (GR1 hi CD11bhi) at both time points, as well as reductions in the percentage and number of infiltrating macrophage/monocytes (GR1lo CD11bhi). Other phenotypic changes included lower percentages of M-CSFR⁺ CD16/CD32⁺ and CD44⁺ cells in the synovial tissues of G-CSF−/− mice with acute arthritis, suggesting that G-CSF may be required for the full induction of these activation markers on synovial cells.

Staining of non-adherent leukocytes after overnight culture also revealed a reduction in CD4⁺ lymphocyte infiltration at day 7, suggesting that G-CSF is required for trafficking of not only neutrophils and macrophages into the joint but also of CD4⁺ T lymphocytes (FIG. 6C).

### EXAMPLE 12

### Depletion of Neutrophils in G-CSF^{-/-} and WT Mice in Acute Inflammatory Arthritis

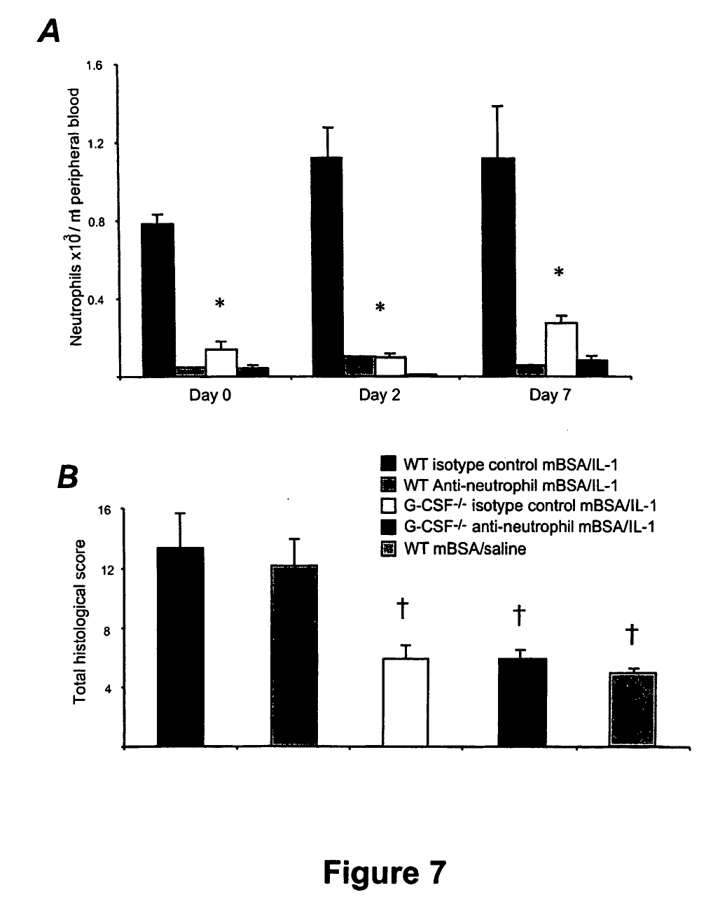

To investigate whether the reduction in mBSA/IL-1-induced arthritis in G-CSF^{-/-} mice was simply a result of neutropenia (Lieschke et al., 1994, supra), neutrophils were depleted using the monoclonal antibody (mAb), RB6.8C5. WT and G-CSF^{-/-} mice were injected intra-peritoneally (i.p.) with anti-neutrophil mAb (RB6.8C5) or isotype control mAb (GL121). Peripheral blood was analyzed for neutrophil levels on days 0, 2 and 7 by differential cell count analysis (FIG. 7A). In WT animals treated with anti-neutrophil mAb, >90% depletion was observed at all times compared to isotype control mAb treated animals (which developed a marked neutrophilia). Neutrophil depletion of WT mice did not abrogate development of arthritis (FIG. 7B), although it did significantly decrease the joint space exudate. In contrast, G-CSF^{-/-} mice were relatively resistant to disease and additional neutrophil depletion did not further reduce disease severity. This indicates that the reduction in neutrophils in the G-CSF^{-/-} mice is not solely responsible for protection from mBSA/IL-1-induced arthritis.

### EXAMPLE 13

### Exogenous G-CSF can Partly Substitute for IL-1 in Acute Inflammatory Arthritis

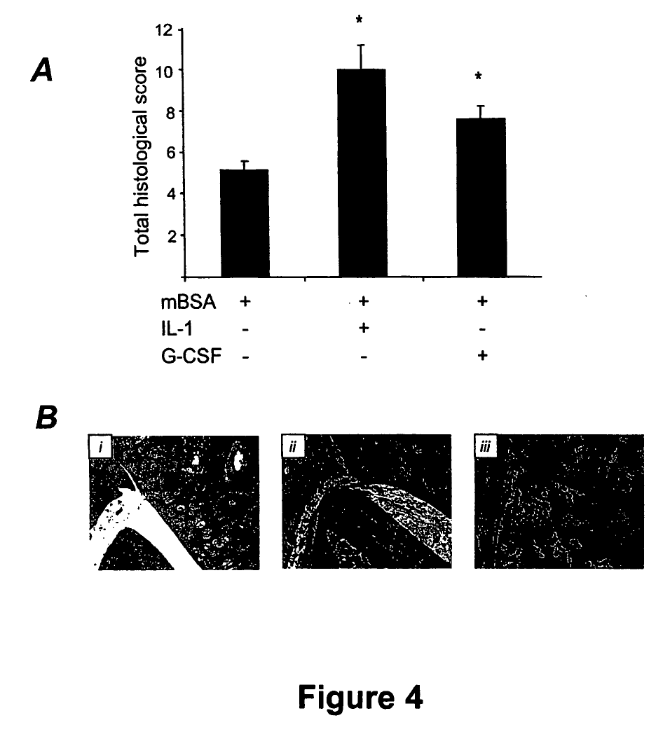

Joints of mice treated with mBSA and G-CSF (mBSA/G-CSF) developed inflammatory and destructive arthritis, although this was less severe than mBSA/IL-1-treated animals (FIG. 4A-B). The major cells infiltrating joints of the mBSA/G-CSF-treated animals were monocyte/macrophages, compared to the predominantly granulocytic infiltrate in mBSA/IL-1-induced arthritis. These results show that systemic administration of exogenous G-CSF can at least partially substitute for systemic IL-1 in driving this model of acute arthritis and that G-CSF leads to the recruitment of monocyte/macrophages into the joint.

### EXAMPLE 14

### G-CSF Deficiency Impairs Collagen-Induced Arthritis (CIA)

G-CSF-Deficient Mice Have Reduced Acute Inflammatory Arthritis

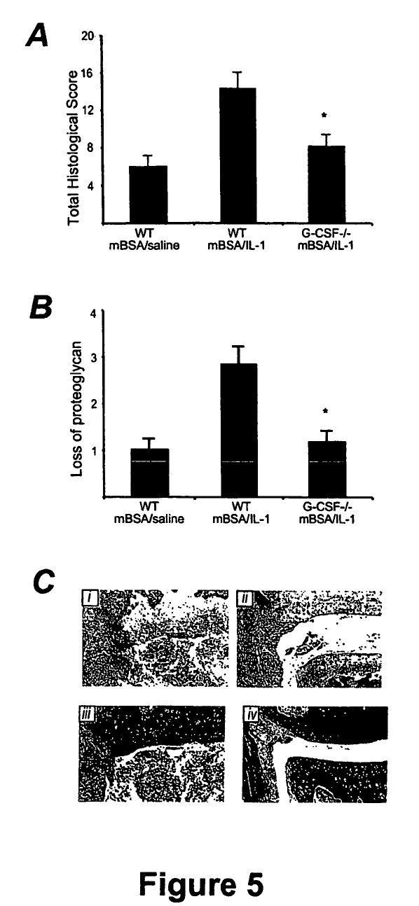

In view of the pro-inflammatory effects of i.a. injection and systemic filgrastim on joint disease, we attempted to determine the absolute dependence of the acute arthritis model on G-CSF, using G-CSF−/− mice. G-CSF−/− and B6 mice were injected i.a. with mBSA (day 0) and s.c. in the footpad with IL-1 on days 0, 1 and 2. Histological assessment of disease at day 7 revealed a significant reduction in inflammatory and destructive features (FIGS. 5A & B). Safranin O staining for cartilage proteoglycan content revealed a major reduction in cartilage proteoglycan loss in G-CSF−/− mice compared to B6 mice (FIG. 5C). Therefore, endogenous G-CSF is an important mediator of inflammation and destruction in this model of acute arthritis.

G-CSF Deficiency Reduces the Incidence and Severity of CIA

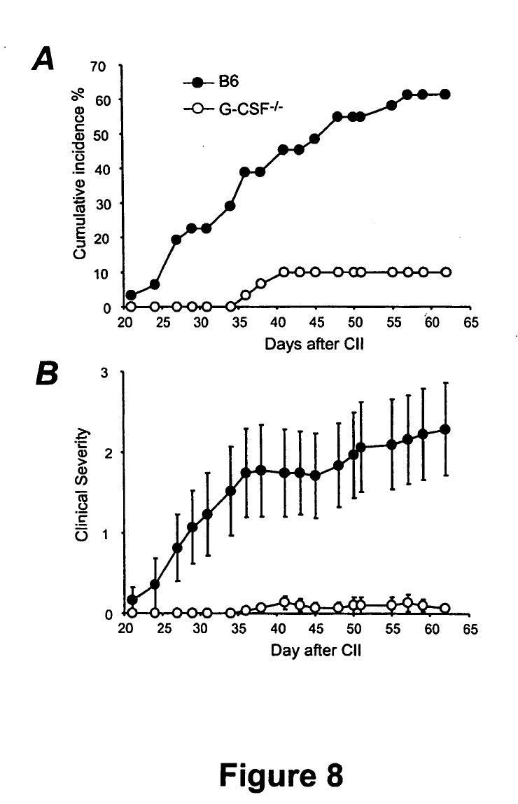

CIA is a chronic autoimmune arthritis that is widely used to study RA. To examine the contribution of G-CSF to CIA, B6 WT and G-CSF−/− mice were immunised with CII in CFA, followed by a boost injection 21 days later (Lieschke et al., Blood 84:1737-46, 1994), and disease incidence and severity compared. The onset of CIA in G-CSF−/− mice was delayed and mice developed disease at a markedly reduced incidence and severity compared to WT mice (FIGS. 8A & B). The reduction of disease incidence and severity in G-CSF−/− mice suggests a pivotal role for endogenous G-CSF in chronic autoimmune arthritis.

### EXAMPLE 15

### Histological Analysis of CIA in G-CSF^{-/-} Mice

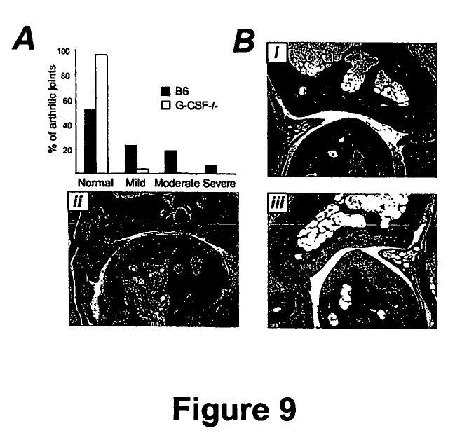

Histological assessment of H&E stained sections of paws from four B6 and G-CSF−/− mice with the highest clinical scores during CIA was performed. Individual joints were scored from 0 normal to 3 (see EXAMPLE 18) and the percentage of normal and arthritic joints determined. There was a significantly greater percentage of normal joints in G-CSF−/− mice compared to WT mice (FIG. 9A), and of the small number of affected paws in the G-CSF−/− mice, none was severe (FIG. 9B). In contrast, joints from WT mice had a range of histological features indicative of mild to severe arthritis (FIGS. 9A & B). These histological observations are concordant with the clinical assessment outlined herein.

### EXAMPLE 16

### In Vitro T Cell Proliferation & Cytokine Production to CII

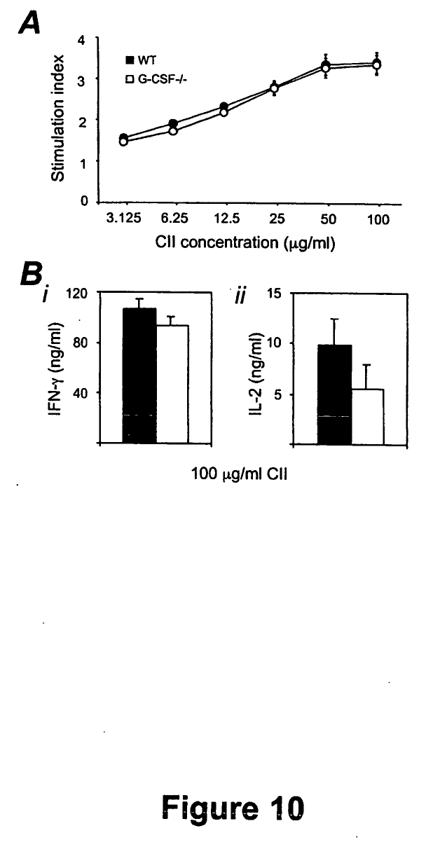

To assess the cellular immune response to CII, in vitro T cell proliferative responses and T cell cytokine (IFNγ and IL-2) production were measured in G-CSF−/− mice and compared to WT. Single cell suspensions were prepared from inguinal LN from mice immunised with CII in CFA and stimulated for 72 h in vitro with 0-100 μg/ml of denatured CII. T cell responses were measured by tritiated TdR uptake in the last 8 h of culture. FIG. 10A depicts the stimulation indices observed in G-CSF−/− and WT mice, which were comparable. Production of T cell cytokines IFN-γ and IL-2 by G-CSF−/− LN cells appeared relatively normal (FIG. 10B).

### EXAMPLE 17

### Impaired Anti-CII Isotype Switching from IgM to IgG in G-CSF−/− Mice

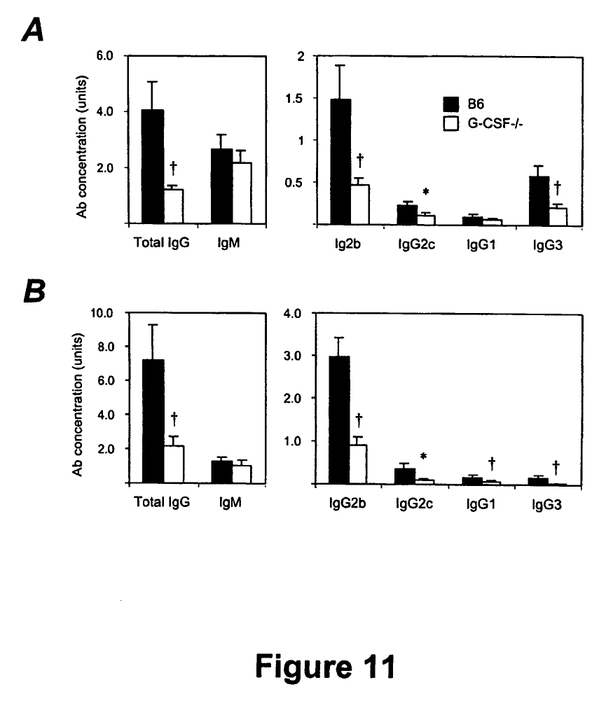

Induction of CIA is dependent on both humoral and cellular immune responses to CII (Campbell et al., 2000, supra). It was examined whether G-CSF deficiency altered serum levels of anti-CII Ab production during CIA (day 30 and 62). Despite comparable levels of anti-CII IgM in WT and G-CSF−/− mice at days 30 and 62, there was a reduction in the level of total anti-CII IgG (FIG. 11). Analysis of anti-CII IgG isotypes revealed reduced production of all isotypes—IgG2b, IgG2c, IgG3 and IgG1. This suggests that there is a defect in isotype switching in G-CSF−/− mice that may contribute to protection against CIA. This observation suggests that endogenous G-CSF plays a role in Ab production by B cells, at least in response to immunisation with antigen in CFA.

### EXAMPLE 18

### Increased Basal Levels of Ig in Naïve G-CSF−/− Mice and Increased Total IgG in CII/CFA-Immunised Mice

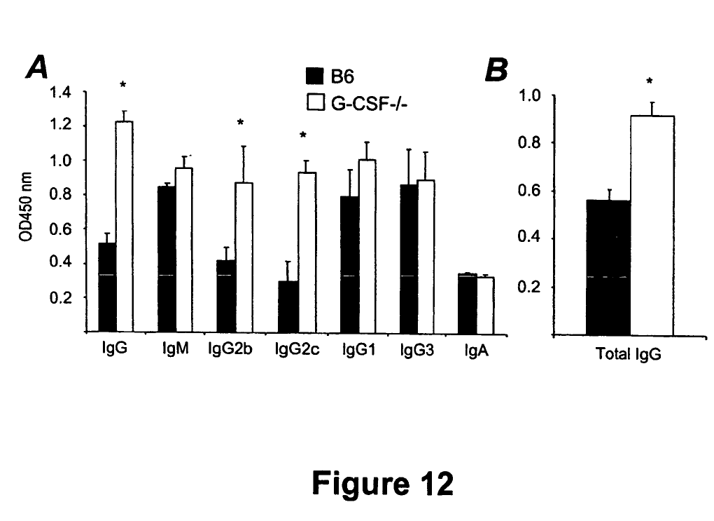

Investigation of the humoral immune response to CII revealed that G-CSF−/− mice have defective switching from an IgM to IgG response. To determine whether this defect reflected pre-existing defects in circulating Ab, sera from naïve mice were analysed for total IgG, IgM and IgG isotypes. Analysis of serum Ab levels revealed significantly greater production of total IgG and isotypes IgG2b and IgG2c in naive G-CSF−/− mice compared to naïve B6 mice and normal baseline production of IgM, IgG1, IgG3 and IgA (FIG. 12A). Furthermore, in CII/CFA-immunised G-CSF−/− mice, levels of non-specific total IgG were markedly enhanced (FIG. 12B). These observations suggest G-CSF plays a role in B cell maturation, antibody production and isotype switching, at least in response to immunisation with antigen in CFA.

### EXAMPLE 19

### Normal T-Dependent and T-independent Ag B Cell Responses in G-CSF−/− Mice

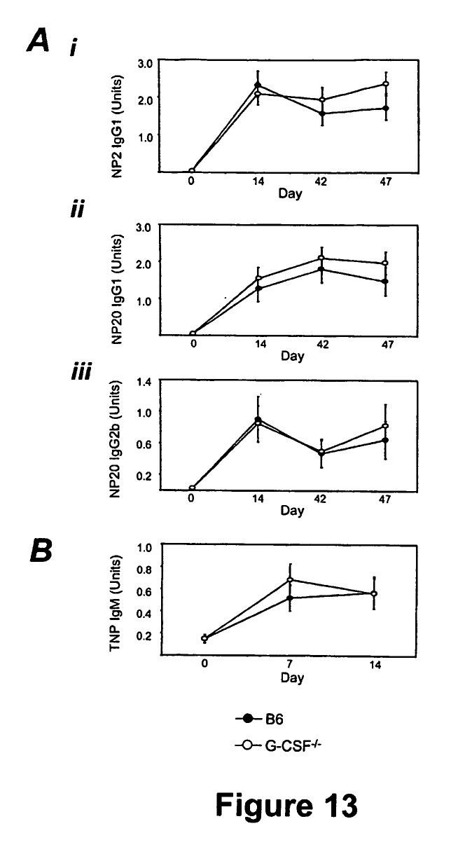

To determine whether the impairment of Ab production in response to CII/CFA was due to a defect in T-dependent or T-independent Ag responses, B6 and G-CSF−/− mice were challenged with the T-dependent antigen NP-KLH in alum, or with the T-independent antigen, DNP-Dextran in PBS. G-CSF−/− mice developed a normal response to both T-dependent and T-independent Ag (FIG. 13), demonstrating that the impaired B cell response in CIA is specific to challenge with CII in CFA.

### EXAMPLE 20

### G-CSF Depletion Causes a Peripheral Blood Neutropenia and Reduces Inflammation in Acute (mBSA/IL-1) Arthritis

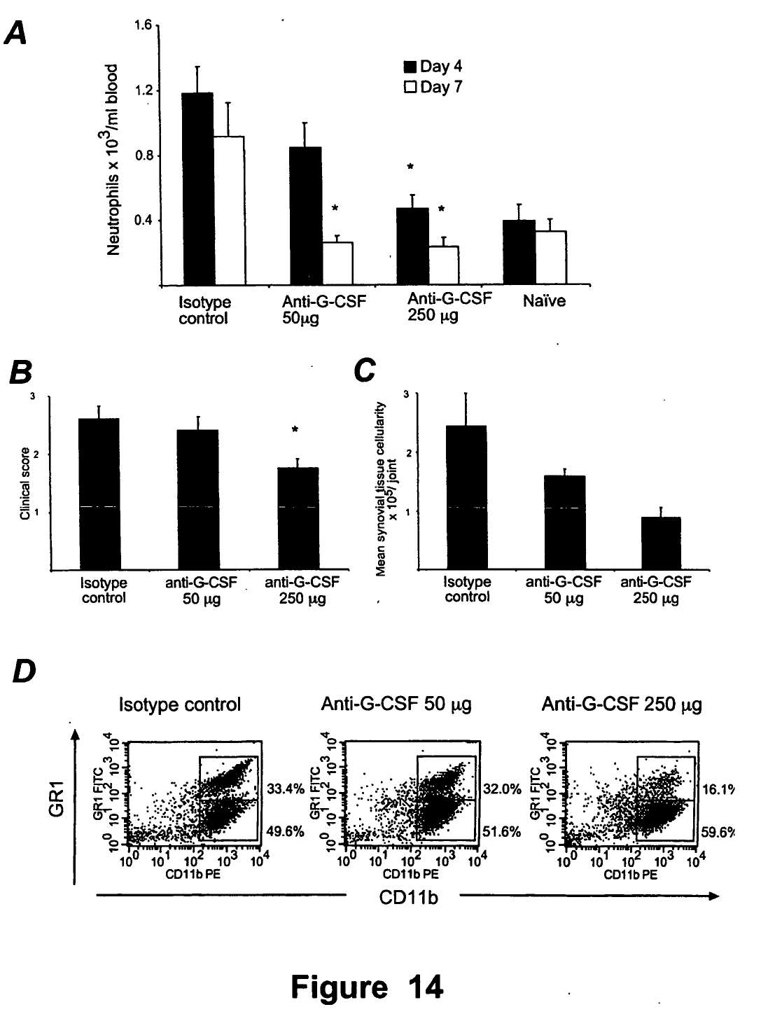

To assess the therapeutic application of G-CSF blockade in WT mice, B6 mice were injected prior to the induction of acute arthritis (day 0) with 50 or 250 μg of rat anti-G-CSF mAb (clone 67604; R&D systems, Minneapolis, Minn., USA) or isotype control (GL113) mAb. mAb was also administered on days 1, 2, 3 and 5. Peripheral blood was taken at days 4 and 7 and subjected to differential analysis (FIG. 14A). Mice receiving the high dose (250 μg) anti-G-CSF mAb had a significant reduction in peripheral blood neutrophils compared to isotype control mAb mBSA/IL-1-treated mice on both days. Mice receiving the lower dose of anti-G-CSF mAb had a significant reduction in neutrophils at day 7 only. Analysis of BM populations also showed a significant reduction in myeloid lineage cells in G-CSF-depleted mBSA/IL-1-treated mice (data not shown). Macroscopic assessment of arthritic joints of mice treated with anti-G-CSF or isotype control mAb revealed a reduction in disease in anti-G-CSF mAb (250 μg) treated mice (FIG. 14B). There was reduced synovial tissue cellularity (FIG. 14C), and a reduced percentage of infiltrating leukocytes, especially neutrophils (GR1hi CD11bhi; FIG. 14D), in mice receiving the higher dose of anti-G-CSF mAb. These results show a dose-dependent reduction in disease features of acute arthritis in response to G-CSF inhibition in wild type mice.

TABLE 2
Bone marrow populations during mBSA/IL-1-induced arthritis

| | | Cells × 10⁴/femur | | | | | | | |
|---|---|---|---|---|---|---|---|---|---|
| | | | | | | | | | Nucleated |
| | Study | | | Promyelocyte/ | Metamyelocyte/ | | | | erythroid |
| Genotype | day | Total cellularity | Blast | Myelocyte | Polymorph | Lymphocyte | Monocyte | Eosinophil | cell |
| B6 | 0 (2) | 2134 ± 103 | 72 ± 2 | 102 ± 4 | 777 ± 4 | 409 ± 18 | 92 ± 2 | 92 ± 7 | 501 ± 2 |
| G-CSF^{-/-} | 0 (2) | 1902 ± 402† | 16 ± 0† | 58 ± 0 | 142 ± 2† | 342 ± 2 | 100 ± 1 | 58 ± 1 | 336 ± 1† |
| B6 | 3 (5) | 1456 ± 79‡ | 23 ± 4‡ | 150 ± 6 | 601 ± 38 | 173 ± 27 | 152 ± 7 | 40 ± 2‡ | 299 ± 24 |
| G-CSF^{-/-} | 3 (5) | 1216 ± 97‡ | 41 ± 6 | 80 ± 7† | 229 ± 21† | 263 ± 18 | 110 ± 13 | 32 ± 4 | 324 ± 28 |
| B6 | 7 (6) | 1710 ± 227 | 21 ± 4‡ | 49 ± 4‡ | 750 ± 14 | 92 ± 12‡ | 99 ± 7 | 54 ± 2 | 213 ± 10‡ |
| G-CSF^{-/-} | 7 (6) | 1458 ± 147 | 34 ± 2‡ | 34 ± 2†‡ | 343 ± 36†‡ | 279 ± 18 | 121 ± 10 | 23 ± 6† | 460 ± 44† |

Cell counts were performed using the Advia counter. Manual leukocyte differentials were performed on diff-quik stained cytospins on cells recovered from one femur per mouse. Values arethe mean ± SD.
*Study day 0 is pretreatment; the value in parentheses is the number of mice studied for that group.
†P < 0.05 for comparison between B6 and G-CSF^{-/-} mice on the same study day
‡P < 0.05 between mice of the same genotype at baseline and on the study day indicated

TABLE 3
Peripheral blood analysis of G-CSF^{-/-} mice during mBSA/IL-1-induced arthritis

| | Day | Cells × 10³ per μl of peripheral blood | | | |
|---|---|---|---|---|---|
| Genotype | of model* | Total WBC | Lymphocytes | Neutrophils | Monocytes |
| B6 | 0 (5) | 3.77 ± 1.00 | 3.17 ± 0.83 | 0.33 ± 0.06 | 0.04 ± 0.02 |
| G-CSF^{-/-} | 0 (4) | 7.51 ± 0.49† | 6.88 ± 0.38† | 0.19 ± 0.03 | 0.03 ± 0.01 |
| B6 | 3 (9) | 2.55 ± 0.30 | 1.89 ± 0.26 | 0.40 ± 0.05 | 0.02 ± 0.00 |
| G-CSF^{-/-} | 3 (9) | 3.15 ± 0.24 | 2.75 ± 0.18† | 0.12 ± 0.03† | 0.03 ± 0.01 |
| B6 | 7 (9) | 3.68 ± 0.80 | 2.68 ± 0.66 | 0.63 ± 0.11 | 0.06 ± 0.02 |
| G-CSF^{-/-} | 7 (9) | 3.28 ± 0.38 | 2.93 ± 0.33 | 0.20 ± 0.04† | 0.03 ± 0.00 |

Peripheral blood was analysed using the Advia counter and cell counts × 10³ per μl determined. Data are represented as mean ± SEM.
*Study day 0 is pre-treatment baseline counts; number in parenthesis is the number of mice examined for that group.
†P < 0.05 between groups on time point examined.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPY

Bungart et al., British Journal of Haematology 22: 1156, 1990;
Campbell et al., Journal of Leukocyte Biology 68: 144-150, 2000;
Campbell et al., European Journal of Immunology 30: 1568-1575, 2000;
Colotta et al., Blood 80: 2012-2020, 1992;
Demetri and Griffin, Blood 78: 2791-2808, 1991;
Douillard and Hoffman, Basic Facts about Hybridomas, in Compendium of Immunology Vol. II, ed. by Schwartz, 1981;
Foster et al., Transplantation 59: 1557, 1995;
Franzke et al., Blood 102: 734-39, 2003;
Gericke et al., Journal of Leukocyte Biology 57: 455-461, 1995;
Gorgen et al., Journal of Immunology 149: 918, 1992;
de Haan et al., Blood. 86: 2986-2992, 1995;
Hamilton et al., Blood 79: 1413-1419, 1992;
Jacob et al., Blood 92: 353-361, 1998;
Kitching et al., Journal of the American Society of Nephrology 13: 350-358, 2002;
Kohler and Milstein, Nature 256: 495-499, 1975;
Kohler and Milstein, European Journal of Immunology 6: 511-519, 1976;
Lawlor et al., Arthritis and Rheumatism 44: 442-450, 2001;
Leizer et al., Blood 76: 1989-1996, 1990;
Lieschke et al., Blood 84: 1737-1746, 1994;
Lord et al., Proc. Natl. Acad. Sci. USA 86: 9499-9503, 1989;
Metcalf, International Journal of Cancer, 25: 225, 1980;
Miyahara et al., Clinical Immunology and Immunopathology 69: 69-76, 1993;
Nakamura et al., Clinical and Experimental Rheumatology 18: 713-718, 2000;
Nicola et al., Nature 314: 625, 1985;
Nicola et al., Journal of Biological Chemistry 258: 9017, 1983;
Pan et al., Blood 86: 4422, 1995;
Rex et al., Transfusion 35: 605-611, 1995;
Roberts et al., Blood 89: 2736-2744, 1997;
Sambrook et al., Molecular Cloning—A Laboratory Manual, Cold Spring Harbour, New York, USA, 1990;
Snowden et al., Bone Marrow Transplantation 22: 1035-1041, 1998;
Souza et al., Science 232: 61, 1986;
Takahashi et al., Laboratory Investigation 74: 827-834, 1996;
Tanabe et al., Rheumatology International 16: 67-76, 1996;
Welte et al., Blood 88: 1907-1929, 1996;
Xu et al., British Journal of Haematology 93: 558-568, 1996;
Yong and Linch, European Journal of Haematology 49: 251-259, 1992;
Yong, British Journal of Haematology 94: 40-47, 1996;
Zavala et al., Journal of Immunology 163: 5125-5132, 1999;
Zavala et al., Journal of Immunology 168: 2011-2019, 2002;

1. A method for the treatment or prophylaxis of arthritis in a subject, said method comprising administering to the subject an effective amount of an agent which inhibits the activity of granulocyte-colony stimulating factor (G-CSF) or a functional or structural homolog thereof or granulocyte-colony stimulating factor receptor (G-CSFR) or a structural or functional homolog thereof and/or which reduces the level of expression of a gene encoding said G-CSF or G-CSFR.

2. The method of claim 1 wherein the arthritis is chronic inflammatory arthritis.

3. The method of claim 1 wherein the condition is rheumatoid arthritis (RA).

4. The method of claim 1 wherein the arthritis is collagen induced arthritis (CIA).

5. The method of claim 1 wherein the subject is an animal or avian species.

6. The method of claim 5 wherein the animal is a mammal.

7. The method of claim 6 wherein the mammal is a primate.

8. The method of claim 7 wherein the primate is a human.

9. The method of claim 6 wherein the mammal is a rodent.

10. The method of claim 9 wherein the rodent is a mouse.

11. The method of claim 1 wherein the agent is an antibody raised against G-CSF or G-CSFR.

12. The method of claim 11 wherein the antibody is a monoclonal antibody.

13. The method of claim 11 wherein the antibody is a polyclonal antibody.

14. The method of claim 1 wherein the agent is soluble G-CSFR or a functional homolog, analog or derivative thereof.

15. The method of claim 1 wherein the agent is a chemical analog of G-CSF.

16. The method of claim 1 wherein the agent is a protein.

17. The method of claim 14 wherein the agent is a protein.

18. The method of claim 1 wherein the gent is a nucleic acid.

19. The method of claim 18 wherein the nucleic acid is DNA or RNA and comprises a sense or antisense polynucleotide sequence or a genetic sequence encoding G-CSF or G-CSFR or part or transcript thereof.

20. The method for identifying an agent which inhibits the activity of G-CSF or G-CSFR said method comprising contacting putative inhibitory agents with said G-CSF or G-CSFR, wherein the agent is identified as an inhibitory agent by binding or otherwise associating with G-CSF or G-CSFR.

21. The method for identifying an agent which regulates the expression of a genetic sequence encoding G-CSF or G-CSFR said method comprising contacting putative regulatory agents with said genetic sequence encoding a G-CSF or G-CSFR, wherein the agent is identified as a regulatory agent by binding or otherwise associating with said genetic sequence encoding a G-CSF or G-CSFR.

22. A pharmaceutical composition comprising an agent which inhibits the activity of G-CSF or G-CSFR in a subject and/or which reduces the level of expression of the gene encoding said G-CSF or G-CSFR in a subject, together with a pharmaceutically acceptable carrier or diluent.

23. The pharmaceutical composition of claim 22 wherein the subject is an animal or avian speicies.

24. The pharmaceutical composition of claim 23 wherein the animal is a mammal.

25. The pharmaceutical composition of claim 24 wherein the mammal is an primate.

26. The pharmaceutical composition of claim 25 wherein the primate is a human.

27. The pharmaceutical composition of claim 26 wherein the mammal is a rodent.

28. The pharmaceutical composition of claim 27 wherein the rodent is a mouse.

29. The pharmaceutical composition of claim 22 wherein the agent is an antibody raised against G-CSF or G-CSFR.

30. The pharmaceutical composition of claim 29 wherein the antibody is a monoclonal antibody.

31. The pharmaceutical composition of claim 29 wherein the antibody is a polyclonal antibody.

32. The pharmaceutical composition of claim 22 wherein the agent is soluble G-CSFR or a functional homolog, analog or derivative thereof.

33. The pharmaceutical composition of claim 22 wherein the agent is a chemical analog of G-CSF.

34. The pharmaceutical composition of claim 22 wherein the agent is a chemical analog of G-CSFR.

35. The pharmaceutical composition of claim 32 wherein the agent is a protein.

36. The pharmaceutical composition of claim 22 wherein the agent is a nucleic acid.

37. The pharmaceutical composition of claim 36 wherein the nucleic acid is DNA or RNA and comprises a sense or antisense polynucleotide sequence or a genetic sequence endcoding G-CSF or G-CSFR, or fragments thereof, flanking a positive or negative selectable marker.

38. A targeting or marker-exchange mutagenesis vector useful for inactivating a gene encoding G-CSF or G-CSFR in a cell, said vector comprising two segments of genetic material encoding G-CSF or G-CSFR, or fragments thereof, flanking a positive or negative selectable marker.

39. A genetically modified animal cell comprising the vector of claim 35 or part of said vector.

40. The genetically modified cell of claim 39 wherein said cell is an embryonic stem cell.

41. A genetically modified animal or embryo comprising, being derived from, one or more of the cells of claim 39, wherein said animal produces low amounts of G-CSF or G-CSFR relative to a non-genetically modified animal of the same species.

42. The genetically modified animal cell of claim 39 wherein the animal is a mouse.

43. The genetically modified animal of claim 42 wherein the animal is a human.

44. A method of producing the genetically modified cell of claim 39, said method comprising introducing a vector comprising two segments of genetic material encoding G-CSF or G-CSFR, or fragments thereof flanking a positive or negative selectable marker, into one or more embryonic stem (ES) cell(s) and selecting for expression of the selectable marker gene, wherein the G-CSF and/or G-CSFR gene in the resultant transformed cell(s) is inactivated by homologous recombination with said vector.

45. A in-vivo method for identifying agents capable of inhibiting the activity of G-CSF and/or inhibiting the interaction of G-CSF with G-CSFR and thereby ameliorate the effects of arthritis, said method comprising administering a putative inhibitory agent to an animal, wherein said agent is identified as having interactivity with G-CSF or G-CSFR.
